(19) United States
(12) Patent Application Publication
(10) Pub. No.: US 2026/0147075 A1
(43) Pub. Date: May 28, 2026

(54) STATISTICAL ABSORPTION ESTIMATION

(71) Applicant: Siemens Healthineers AG, Forchheim (DE)

(72) Inventor: Uvo Hölscher, Erlangen (DE)

(21) Appl. No.: 19/399,565

(22) Filed: Nov. 24, 2025

(30) Foreign Application Priority Data
Nov. 25, 2024 (DE) 10 2024 211 273.9

(51) Int. Cl. G01R 33/56; A61B 5/055

(52) U.S. Cl. CPC G01R 33/5608; A61B 5/055; A61B 2576/00

(57) ABSTRACT

The invention relates to a method for predicting absorption in medical imaging by providing (S1) an absorption model that is based on at least one physiological parameter and a tissue distribution, providing (S3) a group of patient data, translating (S4) the group of patient data into a statistical distribution of at least the physiological parameter and determining (S5) a statistical distribution of absorption values for the group with the absorption model in dependence upon the statistical distribution of the physiological parameter.

This application claims the benefit of German Patent Application No. DE 10 2024 211 273.9, filed on Nov. 25, 2024, which is hereby incorporated by reference in its entirety.

### BACKGROUND

The present embodiments relate to a method for predicting absorption in medical imaging. In addition, the present embodiments relate to an apparatus for predicting absorption and also to a medical imaging system and a computer program.

In magnetic resonance (MR) imaging, specific absorption rate (SAR) describes the fact that the irradiated high-frequency fields (B1) not only excite the spins, but are also absorbed by the tissue and thus deposit energy in the tissue. This causes the tissue to heat up. IEC standard 60601-2-23 therefore specifies limit values for a maximum SAR. For example, the head may have a maximum average SAR of 3.2 W/kg within a 360 second window.

One characteristic of SAR is that it is patient-dependent. The energy that is deposited in the tissue does not depend on the magnetic component (e.g., B field; B1) of the transmission field, but on the accompanying electrical component (e.g., E field). According to Maxwell equations, there is a corresponding E field for each dynamic B-field. Unfortunately, the E field is not directly proportional to the B field, but is mainly determined by the position of the patient and the composition of the tissue (e.g., fat or muscle). A SAR model (hereinafter generally referred to as the absorption model) is required here, which converts the well-known spatial distribution and strength of the B1 field (e.g., B1 defines the flip angle) into an E field and ultimately into an absorbed power per kg of body tissue. This model may take into account the patient's height, weight and, if applicable, age, and thus scales typical body models in which the tissue distribution is stored. Information about the irradiated power of the transmitter coil is also to be provided.

All common MR scanners therefore implement SAR monitoring (e.g., based on the SAR model) that prevents the SAR limits from being exceeded. However, from a workflow point of view, it is desirable that the SAR monitoring does not simply interrupt an ongoing scan in the middle of the measurement when the limits are exceeded, but that the system already detects before the measurement whether the following measurement may be performed within the SAR limits. Therefore, it is also common for a simulation/estimation to be made before each measurement as to whether the following measurement exceeds limits. If this is the case, the scanner may inform the user and, if necessary, even make suggestions for reducing the SAR. Most of these suggestions are either associated with a longer measurement time (e.g., greater TR, pause after the measurement) or with poorer image quality (e.g., reduced flip angle). Therefore, these measures are not used as standard, but an attempt is made to use the measures as little as possible.

This method works well as long as a specific individual case is always considered. In individual cases, values such as body weight, age, irradiated power, etc. are known, since the values are either entered during patient registration or because the values are measured by adjustment steps.

If a protocol (e.g., examination protocol) is planned without a specific patient in the scanner, many of the values for the SAR model are not available. For this reason, the SAR limits are not monitored when “offline editing” a protocol (e.g., editing a protocol without a patient being in the scanner).

“Offline editing” is currently becoming increasingly important, since, for example, many scanners are managed in a fleet association, in which the protocols are edited centrally.

When “editing online” (e.g., with the patient in the scanner), there is also the problem that a protocol below the SAR limits is not yet a guarantee that it will work with all the following patients due to the patient dependence on SAR.

There is a fundamental problem that there is no statement about the SAR behavior of a protocol across an ensemble of patients and scanners.

So far, this problem has been solved in two ways:

The users who set up protocols for a scanner/fleet scan the protocol multiple times for testing and thus get a feel for whether the SAR remains below the limits in a majority of patients. This may be an iterative task, in which protocols are repeatedly adapted and reach a target state over time.

It is also accepted that protocols exceed SAR limits and thus gives the user of the scanner the task of adapting the protocol to the patient during the examination every time it is exceeded (e.g., usually detected by a prediction). This may be efficient if the adjustment only needs to be made in a few patients. However, it may also be very inefficient if the adjustments are made frequently or if the overall duration of an examination may be planned worse as a result.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, absorption in medical imaging may be better predicted.

In accordance with the present embodiments, a method for predicting absorption in medical imaging is provided. The absorption may generally relate to electromagnetic radiation or electromagnetic fields. For example, in medical imaging, a corresponding radiation or a corresponding field is irradiated into a patient to be examined. The patient's tissue partially absorbs the irradiated energy. Unabsorbed energy may be used for imaging. Medical imaging may happen on magnetic resonance technology, X-ray technology, ultrasound technology, and the like.

In one act of the method in accordance with the present embodiments, an absorption model that is based on at least one first physiological parameter and a tissue distribution is provided. The absorption model may include, for example, a body model with which the geometry of a respective body may be scaled (e.g., based on height and weight). Other input parameters of the respective model may be other physiological parameters such as age, gender, etc. It is also possible using the absorption model to simulate the tissue distribution in a virtual body. It is possible using this tissue distribution to determine local absorption values. Altogether, an absorption model for an input parameter combination of, for example, age, weight, and gender may be used to determine a spatial absorption distribution.

In a further act of the method in accordance with the present embodiments, a group of patient data is provided. The patient data may be selected, for example, from a database. The data may be provided on a data carrier or a storage facility or in a network. The patient data that is provided may be received, read, and/or preprocessed for further processing. The group of patient data provides that a data set exists for each individual patient. A group of patient data thus corresponds to a plurality of patient data sets.

In a further act of the method in accordance with the present embodiments, the group of patient data is translated into a statistical distribution of at least the first physiological parameter. For example, a selection parameter for selecting a group of patient data contains the value “male.” The gender as a selection parameter may now be translated into a statistical distribution of the height of the respective patient. In this example, the variable corresponds to the first physiological parameter. Similarly, the group of selected patient data may also be translated into a statistical distribution of the body weights of the patients. In general, the group of patient data may be translated into multiple statistical distributions, such as with regard to age or another physiological parameter.

In a further act of the method in accordance with the present embodiments, a statistical distribution of absorption values for the group with the absorption model is determined in dependence upon the statistical distribution of the first physiological parameter. For example, the statistical distribution of the absorption values is determined by calculation with the aid of the absorption model. The statistical distribution of the first physiological parameter is used as an input variable of the absorption model for the determination. Irrespective of this, one or more other statistical distributions or individual values may also be used as input parameters. This provides that at least one of the input parameters of the absorption model is a statistical distribution. It is thus possible to obtain an absorption distribution for a group of patient data or patients. For example, a statement about the SAR behavior of a protocol may be made regarding an ensemble of patients.

In one example embodiment, the group of patient data is selected from a database by setting a first value range of a first physiological selection parameter as the selection criterion. For example, patients between the ages of 18 and 65 are selected. In this case, the first physiological selection parameter corresponds to the age of a respective patient. In principle, however, the value range may also relate to another physiological selection parameter, such as weight or height. The value range is defined by a minimum value and a maximum value. If, for example, the physiological selection parameter is gender, the value range may include only a single value, so that in this case, the minimum value and the maximum value are the same.

The database contains numerous patient data or patient data sets that are selected according to the selection criterion. If necessary, the selection does not provide a patient data set, only one patient data set, or many patient data sets. A user of medical imaging may thus focus on a specific group of patients.

In accordance with a further example embodiment, the group of patient data is selected from the database by setting a second value range of a second physiological selection parameter, which is different than the first, as an additional selection criterion. Two dimensions are thus used for the selection of the group of patient data, where the first dimension is the first physiological selection parameter and the second dimension is the second physiological selection parameter. For example, the first dimension or the first selection parameter relates to the age, and the second dimension or the second selection parameter relates to the weight of the patients. Optionally, further dimensions or selection parameters may also be used to select the group of patient data. As already indicated above, the height and gender of the patients, for example, may also be used as selection parameters. Using such a multidimensional selection method (e.g., combination of value ranges of physiological parameters), a group of patient data may be selected very specifically.

In a further example embodiment, it is provided that the group of patient data is additionally translated into a statistical distribution of a second physiological parameter, which is different than the first physiological parameter, as input parameter of the absorption model for determining the statistical distribution of the absorption values. The selected group of patient data is thus not only translated into a first statistical distribution of the first physiological parameter, but also into a second statistical distribution of a second physiological parameter (e.g., multidimensional). The group of patient data may also be translated into a third and further statistical distribution that is different than the first two statistical distributions. In a specific example, the group of patient data may thus be translated into a first statistical distribution of the weight of the patients and into a second statistical distribution of the age of the patients. Optionally, the selected group of patient data may also be translated into a third statistical distribution of the gender of the patients. These statistical distributions that are now available may be used as input parameters for the absorption model. In this manner, a statistical distribution of absorption values may be calculated using the absorption model in dependence upon the different distributions of the different physiological parameters.

In a further example embodiment, the group of patient data is additionally translated into a statistical distribution of a patient-specific and device-specific adjustment parameter for an apparatus, which is used for medical imaging, as an input parameter of the absorption model for determining the statistical distribution of the absorption values. Typically, medical imaging apparatuses (e.g., magnetic resonance apparatuses) are to be adjusted specifically for patients. A corresponding adjustment value is to usually be measured in advance for each patient. This is done, for example, using a short measurement before the actual examination. The adjustment value or adjustment parameter is not only patient-specific, but also device-specific. This provides, for example, that in the case of different MR devices (e.g., magnetic resonance devices), different adjustment values are also measured. In the database, a corresponding adjustment value may thus be present for a plurality of magnetic resonance imaging devices for a plurality of or all patients. The distribution of the absorption values may now be calculated in dependence upon these adjustment values or a distribution of these adjustment values. In a fleet scenario (e.g., fleet of different imaging devices), a prediction may thus be made for each individual scanner or for an ensemble of scanners. For example, it may be predicted that one or more scanners of a fleet will exceed a SAR limit and other scanners will not.

In a further example embodiment, it is provided that the first physiological parameter, the second physiological parameter, the first physiological selection parameter, and/or the second physiological selection parameter is in each case selected from the group:
age, weight, gender, and body region to be examined. Both the selection of the patient or patient data and the input into the absorption model (e.g., input parameters) may thus be based on various physiological parameters. For example, a group of patients between the ages of 20 and 50 of the male sex and a height between 1.70 and 2 meters may be selected, in which the knee was always examined as a body region. In a similar manner, other combinations of physiological parameter values may also be used for the selection.

In a further example embodiment, it is provided that when the statistical distribution of absorption values for the group is being determined, a statistical parameter of the statistical distribution of the second physiological parameter is used as an input parameter for the absorption model. The statistical distribution of the first physiological parameter is thus used as an input parameter for the absorption model, but instead of the second statistical distribution of the second physiological parameter, a statistical parameter of this second statistical distribution is used. For example, an average value or a start value of a percentile is used as a representative parameter. Thus, a simulation of the absorption values does not have to be performed for all combinations of input parameters of the statistical distributions. Rather, the simulation may be reduced to representative combinations.

According to a further example embodiment, when the statistical distribution of absorption values is being determined, a history of absorption events in a fixedly defined time window is taken into account. The energy that is deposited in a volume element by irradiation is dissipated again over the course of time by heat emission. Therefore, for example, a SAR limit value is exceeded by slight irradiation if the SAR limit value was almost reached in a preceding sequence section. It is therefore important that the history of absorption events is observed in a fixedly defined time window. The standard IEC 60601-2-23 defines specific time windows of 10 s and 360 s. By taking the time windows into account, it may be prevented that SAR limit values are exceeded by unfavorable temporal placements of individual sequences.

In accordance with a further example embodiment, the statistical distribution of absorption values is predicted separately for each partial volume of an examination region. Thus, for example, a spatial distribution may be determined that indicates for each partial volume in the examination region how the absorption distribution is estimated there. For example, the color red may be used for displaying this statistical distribution so that the SAR limit value is exceeded with a probability of more than 80%. For example, 1 cm³ may be selected for a size of the partial volumes. However, coarser or finer resolutions may also be used.

In a further example embodiment, the statistical distribution of absorption values is compared with a limit value, and corresponding comparison information is provided. The statistical distribution indicates the probability with which a respective absorption value occurs. If this distribution is now compared with a limit value, it may be determined, for example, with which probability an absorption value exceeds this limit value. For example, it may be helpful for a user of an MR imaging system if the user knows that a certain protocol for a head examination in less than 10% of the patients exceeds the prescribed limit value. The comparison of the respective absorption value with the limit value provides comparison information. This comparison information is provided and may be further processed. For example, the comparison information may be used for a color coding or a warning.

In a specific example embodiment, a proportion of the statistical distribution of the absorption values that is above or below the limit value is calculated. For example, an absorption value, optionally resolved by partial volumes, is determined for all possible combinations of the input parameters of the absorption model or only for a part of these combinations. This results in a multidimensional distribution of the absorption values. Via this distribution, it is now possible to integrate those individual combinations that are above or below a predetermined limit value. Thus, for example, the proportion of those patients from the selected group in which the limit value is exceeded or undershot may be determined.

In an example embodiment, the medical imaging is based on magnetic resonance technology. In principle, however, the method may also be used for other technologies such as X-ray technology or ultrasound technology. In any case, a prediction may be made reliably as to whether any limit values are exceeded by the energy that is irradiated in each case.

In accordance with a further example embodiment, the absorption values each represent a specific absorption rate. Specifically, an absorption value may thus represent a SAR value. Such SAR values are necessary in imaging methods in which energy is introduced into an object to be examined, whether by radiation or in another physical way.

In a further example embodiment, it is provided that the determination of the distribution of the absorption values is performed using an absorption model in dependence upon a temporal distribution of a high-frequency field. Especially in magnetic resonance examinations, a high-frequency field (B1 field) is irradiated into the object to be examined. Such a high-frequency field is time-variable and is composed of high-frequency pulses. The temporal distribution of this high-frequency field may be used as an input variable for the absorption model. Thus, the determination of the absorption values may be matched very specifically to the temporal distribution of the high-frequency field. Thus, for example, the repetition times of an examination protocol are reflected in the correspondingly predicted absorption values.

In a further example embodiment, a proposal for a change in a parameter of an examination protocol of the medical imaging is automatically determined in dependence upon the comparison information. If, for example, the comparison information represents the case that a limit value is exceeded in the absorption, it is advantageous if it is automatically proposed by the imaging system that a parameter of the examination protocol should be changed. For example, it may be proposed that the repetition time be extended from 2 to 2.7, since, for example, the limit value is exceeded in 10 percent of the patients. By extending the repetition time, the pauses between the measurements are increased, so that the energy that is introduced may be better dissipated.

As another example, an apparatus for predicting an absorption value in medical imaging is provided. The apparatus includes a data processing apparatus (e.g., including one or more processors) that is configured so as to implement a method as described above. The advantages and further development possibilities described above in connection with the method in accordance with the present embodiments also apply mutatis mutandis to the apparatus in accordance with the present embodiments. The method features that are described may accordingly be interpreted as functional features of the apparatus.

In addition, a medical imaging system having such an apparatus may also be provided. Such an imaging system may be, for example, a magnetic resonance system, an X-ray system (e.g., CT system or angiography system), or a sonography system.

Further, a computer program or computer program product is provided that includes commands that, when executed by the aforementioned apparatus, trigger the apparatus to implement the method also mentioned above. For example, the computer program may be executed by a computing facility that has one or more processors and one or more storage facilities.

### BRIEF DESCRIPTION OF THE DRAWINGS

FIG. 1 shows a schematic representation of an example embodiment of an imaging system;

FIG. 2 shows a distribution of SAR values over combinations of physiological values; and

FIG. 3 shows a schematic flow chart of an example embodiment of a method for predicting an absorption.

### DETAILED DESCRIPTION

The embodiments described in more detail below represent examples of the present embodiments.

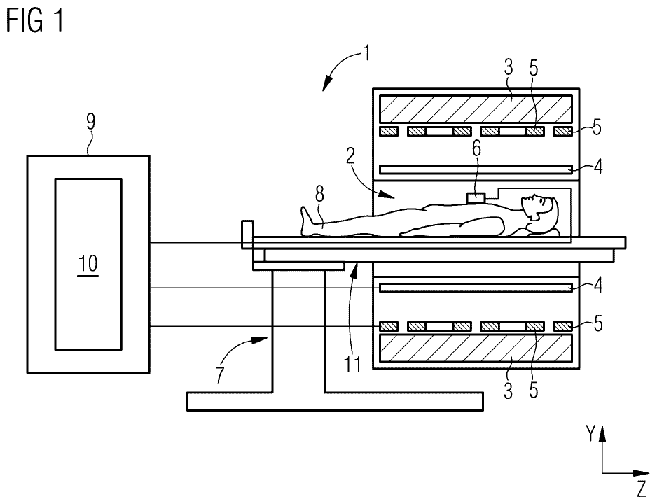

FIG. 1 illustrates a specific embodiment of a magnetic resonance tomography (MRT) system 1.

The MRT system 1 includes a magnet unit having a field magnet 3 that generates a static magnetic field for orienting nuclear spins of an object 8 (e.g., a patient) in an imaging region. The imaging region or examination region is characterized by an extremely homogeneous static magnetic field, where the homogeneity relates, for example, to the magnetic field strength or an amplitude of the magnetic field. The imaging region is located in a patient tunnel 2 that extends in a longitudinal direction Z through the magnet unit. The field magnet 3 may, for example, be a superconducting magnet that may generate magnetic fields having a magnetic flux density of up to 3 T or more. However, permanent magnets or electromagnets having normally conducting coils may also be used for lower field strengths. An object transport unit configured, for example, as a patient table 7 has a patient couch 11 that may be moved into the patient tunnel 2 in order to transport the part of the patient (e.g., object) to be examined into the examination region within the patient tunnel 2.

Further, the magnet unit includes a gradient coil arrangement 5 having a plurality of gradient coils that serve to superimpose gradient fields (e.g., location-dependent magnetic fields) on the static magnetic field in the three spatial directions in order to spatially differentiate the scanned image regions in the imaging region. The gradient coils of the gradient coil arrangement 5 may be configured, for example, as coils of normally conducting wires that may generate mutually orthogonal fields or field gradients in the imaging region, for example.

The magnet unit includes a transmission coil arrangement that may include, for example, a body coil 4 as a transmission antenna that is configured so as to radiate a high-frequency signal into the imaging region. The body coil 4 may therefore be understood as an HF transmission coil arrangement of the MRT system 1 or as part of the HF transmission coil arrangement. In some embodiments, the body coil 4 may also be used to receive resonant MR signals that are emitted by the object 8. In this case, the body coil 4 may also be considered as part of a signal detection apparatus of the MRT system 1. Optionally, the signal detection apparatus includes a local coil 6 that may be arranged in the immediate vicinity of the object 8 (e.g., on the object 8 or in the patient table 7). As an alternative or in addition to the body coil 4, the local coil 6 may be used as a receiving coil or receiving antenna.

The MRT system 1 also includes a control and computing system 9. The control and computing system 9 may include a transceiver control unit 10 that is connected to the body coil 4, the gradient coil arrangement 5, and/or the local coil 6. Depending on the detected MR signals, the transceiver control unit 10, which may include an analogue-digital converter ADC, may generate corresponding MR data (e.g., in the k-space). The transceiver control unit 10 is optionally also connected to the body coil 4 and controls the body coil 4 to generate HF pulses, such as excitation pulses and/or refocusing pulses. Further, the transceiver control unit 10 of the control and computing system 9 may also be connected to the gradient coil arrangement 5 and control the gradient coil arrangement 5 in order to switch slice selection gradients, gradients for the frequency, and/or phase coding, and/or read-out gradients.

The control and computing system 9 may evaluate the MR data and, for example, perform an image reconstruction or parts thereof or other computing tasks that are necessary in the course of the imaging. A digital signal processing facility may be part of the transceiver control unit 10 and thus also part of the examination system (e.g., MRT system 1).

The described structure of the control and computing system 9 is only a non-limiting example. The various required tasks and functions may also be different and/or distributed to different control units and/or other computing units.

The control and computing system 9 of the MRT system 1 is, for example, capable of implementing a method in accordance with the present embodiments for predicting absorption (e.g., of an electromagnetic field) in medical imaging.

There is, for example, a requirement to make a statement about the specific absorption rate (SAR) behavior of a protocol across an ensemble of patients and/or scanners.

When “offline editing” a protocol, the SAR prediction for a specific patient cannot be made (e.g., because patient-specific data is missing), but a prediction may be made for a specific ensemble of patients. This provides that assumptions may be made with regard to this ensemble about the statistical distribution of values relevant to the SAR model (e.g., general absorption model), and statistical statements may be made about the distribution of the expected SAR values (e.g., absorption values). For example, the question may be answered as to how likely it is that for patients between the ages of 18 and 75, a certain protocol in the “head” examination region will exceed a predetermined limit value in less than 10% of the patients. This information is referred to as, for example, “statistical SAR,” in contrast to the specific “patient SAR” (e.g., SAR for a specific patient in a specific scanner).

A “statistical SAR” may be performed, for example, with the following steps 1 to 5, where not all steps are absolutely necessary: 1) For a certain protocol, the temporal distribution of B1² (e.g., the B1 field) should be known. This is determined by flip angle, HF pulse types, and timing between the pulses. This value depends only on the protocol parameters and is not patient-dependent. It is used both in the “patient SAR” and in the “statistical SAR.”

The temporal distribution of B1² is not patient-dependent. In other words, the temporal distribution of B1² only has to be determined once and may then be used for any number of calculations of the protocol with different patient-dependent parameters.

2) The user determines how the target patient group is defined (e.g., the user selects a group of patient data (sets) from a database). This selection using physiological parameters as selection parameters may have the following characteristics: a. Determination of a patient group by, for example, an age range (e.g., the physiological selection parameter is age), where a minimum value and a maximum value may define the age range; b. Determination of a patient group by, for example, the examined body region (e.g., the physiological selection parameter is the body region (e.g., the head or the knee); c. Determination of a patient group by, for example, a clinical condition such as obesity (e.g., the physiological selection parameter may be the body mass index (BMI); and d. Combination of the above.

Presets may be provided, for example, that store definitions from multiple values or value ranges as selection parameters and allow the user to select them faster.

3) The scanner (e.g., the imaging modality or imaging apparatus) translates this selected patient group or the corresponding data sets into a statistical distribution of the patient-dependent values. These values may be taken from a database. The following values may be provided: a. statistical distribution of weight as a physiological parameter; b. statistical distribution of age as a physiological parameter; c. statistical distribution of gender as a physiological parameter; d. statistical distribution of the adjustment values such as transmitter adjustment and typical reflection rate of the transmitter coil. The adjustment values are patient-specific and may be measured for each patient in a short examination.

In a fleet scenario of scanners (e.g., a company has multiple scanners of different types), even the dimension that multiple scanners have different distributions in the adjustment values may be taken into account. In one embodiment, the predictions per scanner or as worst-case results may be made over an ensemble of scanners. For example, it may be stated that with a certain measurement protocol for knee examinations, a SAR limit value is exceeded with a probability of 10% for one of the scanners of a fleet, while the SAR limit is not exceeded for the other scanners of the fleet.

4) The user determines how the statistical values are taken into account in the absorption model or SAR model: a. All combinations of patient-dependent, physiological parameters are simulated, and thus, all possible SAR values are determined. A distribution of all possible SAR values is thus obtained. b. Only very specific, representative combinations of patient-dependent, physiological parameters are simulated. For example, the average value of the distribution or the start of the upper 10% percentile may always be taken, and the SAR values may be simulated for this.

There are correlations between much of the data. For example, there is a clear correlation between age and weight. It is therefore sufficient to use one of the two parameters for the selection of the group of patients or patient data sets. In this way, the calculation effort may be reduced.

SAR is determined over time windows that are defined in the standard (e.g., 10 seconds and 360 seconds). For a complete estimate of whether a protocol will be below the limit (SAR limit value), a history in the time length of these two windows is also to be estimated, which was temporally before the measurement to be simulated. In one embodiment, a blanket assumption (e.g., the previous 360 seconds had exactly 90% of the SAR limit) may be used. Alternatively, statistical data that results from the defined patient group may also be used. For example, the average SAR value of all knee examinations of patients between 18 and 75 years of age may be used.

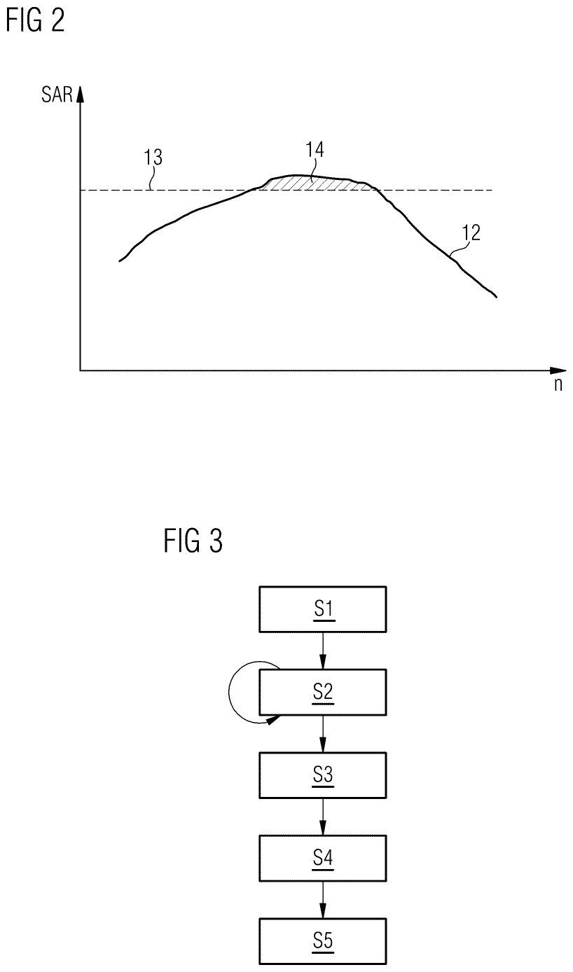

5) The user determines how the results from step 4 are presented. The following characteristics are particularly useful: a. The user is shown how the distribution of the statistical SARs is in relation to the limit values (cf., FIG. 2); b. The user is shown what percentage of all patients in the defined group are statistically above the limit value (cf., FIG. 2); c. The user indicates in which percent of the patients from the defined group they accept a SAR exceedance, and the system shows the user whether this is the case or not; d. A combination of the above possibilities.

The user may determine what percentage of their defined patient group should be below the SAR limit. If this is not the case, it is possible similarly to the solution of a SAR conflict in “patient SAR” for a calculation to be made to suggest which parameters are to be changed and how. For example, the imaging modality or the scanner proposes to extend the repetition time from 2 s to 2.7 s, since 10% of the patients exceed the SAR limit value. A change in another parameter of the imaging modality may also be proposed.

In the “online case” of a patient in the scanner, there are many points in time at which only some, but not all, patient-dependent values are known. Although weight, age, and gender are known immediately after registration, the adjustment values are not yet known. In one embodiment, it is also possible to work with a variant of the “statistical SAR” before the adjustments, where all known values specifically and unknown values are determined statistically. As soon as all patient-dependent values are known, the “statistical SAR” converges toward the “patient SAR”.

FIG. 2 illustrates a distribution of SAR values over a plurality of combinations n. Each combination represents a combination of physiological parameters that characterizes a patient. For example, a combination corresponds to a patient with an age of 25 years, male, weight 80 kilograms, and height 1.8 meters. For all such combinations, the distribution 12 illustrated in FIG. 2 results, for example, for a knee examination. A limit value 13 corresponds, for example, to a SAR value that should not be exceeded. In the present example, this limit value 13 is exceeded by a certain proportion 14 of patients. For this example of FIG. 2, the limit value 13 for a portion 14 of combinations or patients is exceeded with the selected parameters of the imaging apparatus. The user may then decide for themselves whether to retain the settings of the imaging apparatus that is used or, for example, to change the protocol such that the limit value 13 is not exceeded for any patient.

FIG. 3 schematically illustrates a flowchart of an example embodiment of a method. In a first act S1, an absorption model (e.g., a SAR model) that may include a body model is provided. The absorption model may have physiological parameters such as age, weight, gender, etc., as input parameters, as well as one or more adjustment parameters. With the absorption model, a tissue distribution and thus a locally resolved absorption value may be determined in dependence upon an irradiated field. In a second act S2, a value range for a selection parameter is defined in order to select a group of patients or patient data (e.g., representative of patients). Act S2 may be repeated multiple times for different selection parameters. If necessary, an examination region such as the head or knee is also defined in act S2. In act S3, the selection criteria defined in act S2 are applied, for example, to a database in order to extract a corresponding group of patient data therefrom. This group of patient data is provided in a storage facility, for example. In a further act S4, the group of patient data that is provided in act S3 is translated into a statistical distribution of one or more physiological parameters. From the group of patient data, a statistical distribution is determined with regard to one or more physiological parameters such as age, weight, gender, etc. This statistical distribution of a physiological parameter, if necessary combined with the statistical distribution of another physiological parameter, is used as an input space for the absorption model. A corresponding statistical distribution of absorption values is obtained for the group of patient data in act S5.

The advantage of the statistical SAR is that statistical values may be used for the SAR model in the absence of patient-dependent values. These values may be determined from a corresponding database. The statistical data may be limited to a suitable patient ensemble via user input. This results in the following further advantages: a) SAR predictions may be made without a specific patient being in the scanner; b) SAR predictions may be made about a defined patient group, so that it may be controlled much better than in the specific case with a specific patient, at what percentage of all patient measurements SAR values are above the limit, and thus a manual interaction by the user before the measurement will be necessary; c) SAR predictions may be made at any time. In this way, the user may be given a lot of direct feedback on which parameter change has which effect on SAR, both when editing offline and when editing online.

Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for determining a statistical distribution of absorption values in medical imaging, the method comprising:
providing an absorption model that is based on at least one first physiological parameter and a tissue distribution;
providing a group of patient data;
translating the group of patient data into a statistical distribution of at least the at least one first physiological parameter; and
determining the statistical distribution of absorption values for a group with the absorption model in dependence on the statistical distribution of the at least one first physiological parameter.

2. The method of claim 1, wherein providing the group of patient data comprises selecting the group of patient data from a database, the selecting of the group of patient data from the database comprising setting a first value range of a first physiological selection parameter as a selection criterion.

3. The method of claim 2, wherein selecting the group of patient data from the database comprises setting a second value range of a second physiological selection parameter as an additional selection criterion, the second physiological selection parameter being different than the first physiological selection parameter.

4. The method of claim 1, further comprising translating the group of patient data into a statistical distribution of a second physiological parameter that is different than the at least one first physiological parameter, as an input parameter of the absorption model for determining the statistical distribution of the absorption values.

5. The method of claim 1, further comprising translating the group of patient data into a statistical distribution of a patient-specific and device-specific adjustment parameter for an apparatus that is used for medical imaging, as an input parameter of the absorption model for determining the statistical distribution of the absorption values.

6. The method of claim 1, wherein the at least one first physiological parameter, the second physiological parameter, the first physiological selection parameter, the second physiological selection parameter, or any combination thereof is in each case selected from the group comprising age, weight, height, gender, and body region to be examined.

7. The method of claim 4, wherein determining the statistical distribution of absorption values for the group comprises using a statistical parameter of the statistical distribution of the second physiological parameter as an input parameter for the absorption model.

8. The method of claim 1, wherein determining the statistical distribution of absorption values comprises determining the statistical distribution of absorption values taking a history of absorption events in a fixedly defined time window into account.

9. The method of claim 1, wherein the statistical distribution of absorption values is predicted separately for each partial volume of an examination region.

10. The method of claim 1, further comprising:
comparing the statistical distribution of absorption values with a limit value; and
providing corresponding comparison information.

11. The method of claim 10, wherein a proportion of the statistical distribution of the absorption values that is above or below the limit value is calculated.

12. The method of claim 1, wherein the medical imaging is based on magnetic resonance technology.

13. The method of claim 12, wherein the absorption values each represent a specific absorption rate.

14. The method of claim 12, wherein determining the statistical distribution of the absorption values is performed using the absorption model in dependence upon a temporal distribution of a high-frequency field.

15. The method of claim 12, further comprising:
comparing the statistical distribution of absorption values with a limit value;
providing corresponding comparison information; and
automatically determining a proposal for a change in a parameter of an examination protocol of the medical imaging in dependence upon the comparison information.

16. An apparatus for predicting absorption in medical imaging, the apparatus comprising:
a data processing apparatus configured to determine a statistical distribution of absorption values in medical imaging, the data processing apparatus being configured to determine the statistical distribution comprising the data processing apparatus being configured to:
provide an absorption model that is based on at least one first physiological parameter and a tissue distribution;
provide a group of patient data;
translate the group of patient data into a statistical distribution of at least the at least one first physiological parameter; and
determine the statistical distribution of absorption values for a group with the absorption model in dependence on the statistical distribution of the at least one first physiological parameter.

17. A medical imaging system comprising:
an apparatus for predicting absorption in medical imaging, the apparatus comprising:
a data processing apparatus configured to determine a statistical distribution of absorption values in medical imaging, the data processing apparatus being configured to determine the statistical distribution comprising the data processing apparatus being configured to:
provide an absorption model that is based on at least one first physiological parameter and a tissue distribution;
provide a group of patient data;
translate the group of patient data into a statistical distribution of at least the at least one first physiological parameter; and
determine the statistical distribution of absorption values for a group with the absorption model in dependence on the statistical distribution of the at least one first physiological parameter.
